(19) United States
(12) Patent Application Publication
(10) Pub. No.: US 2015/0335848 A1
(43) Pub. Date: Nov. 26, 2015

(54) AUTO-ADJUSTING HEADGEAR FOR A PATIENT INTERFACE DEVICE

(71) Applicant: KONINKLIJKE PHILIPS N.V., Eindhoven (NL)

(72) Inventors: ELIZABETH EURY, LATROBE, PA (US); ERIC ALAN HIGGINS, CHESWICK, PA (US); ROBERT SCOTT DULABON, PITTSBURGH, PA (US); KEVIN DANIEL HIMES, IRWIN, PA (US)

(21) Appl. No.: 14/760,018

(22) PCT Filed: Dec. 23, 2013
(86) PCT No.: PCT/IB2013/061261

§ 371 Date: Jul. 9, 2015

Related U.S. Application Data
(60) Provisional application No. 61/751,002, filed on Jan. 10, 2013.

(51) Int. Cl. A61M 16/06; A61M 16/00

(52) U.S. Cl. CPC A61M 16/0683; A61M 16/0057; A61M 16/0605; A61M 2016/0661; A61M 2205/0216; A61M 2207/00

(57) ABSTRACT

A patient interface device for delivering a flow of breathing gas to an airway of a patient, comprises a mask component and auto-adjusting headgear. The auto-adjusting headgear comprises one or more straps wherein the materials have elasticity characteristics such that the headgear self-adjusts. The headgear comprises two lateral strap sections, a rear strap section, and a top strap section. The strap sections are of varying materials and thicknesses to control the properties of the strap sections. In one embodiment, at least a portion of a strap includes a high friction material disposed so as to contact a portion of the user responsive to the headgear being donned by a user.

### CROSS-REFERENCE TO RELATED APPLICATIONS

This patent application claims the priority benefit under 35 U.S.C. §119(e) of U.S. Provisional Application No. 61/751,002 filed on Jan. 10, 2013, the contents of which are herein incorporated by reference.

### BACKGROUND OF THE INVENTION

1. Field of the Invention

The present invention relates to a patient interface device for transporting a gas to and/or from an airway of a user, and, in particular, to a patient interface device comprising an auto-adjusting self-adjustment mechanism for adjusting mask position and/or pressure.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component comprises, for example, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

For such patient interface devices, a key engineering challenge is to balance patient comfort against mask stability. This is particularly true in the case of treatment of OSA, where such patient interface devices are typically worn for an extended period of time. As a patient changes sleeping positions through the course of the night, masks tend to become dislodged, and the seal can be broken. A dislodged mask can be stabilized by increasing strapping force, but increased strapping force tends to reduce patient comfort. This design conflict is further complicated by the widely varying facial geometries that a given mask design needs to accommodate. One area where facial geometries vary a great deal is the angle of the base of the nose (known as the nasolabial angle).

Another area where fit and comfort are often a concern is the bridge of the patient's nose, as many patient interface devices will apply a pressure to this area. If this pressure is not able to be managed effectively, either a poor fit or patient discomfort, or both, will result, thereby limiting the effectiveness of the device.

Patient interface devices traditionally use fabric headgear straps placed at the top and bottom of a patient's head. These headgear straps are adjusted to fit the majority of CPAP patients and also are tightened to seal the mask as well as to provide stability as the patient moves at night. Typical headgear designs utilize hook and loop and fabric straps to manually adjust the headgear top and bottom straps to fit the variety of patients' head sizes. This type of adjustment tends to result in bulky interfaces due to fabric being folded upon itself, and hard plastic clips are traditionally used to help with the movement of the adjustment.

### SUMMARY OF THE INVENTION

Accordingly, it is an object of the invention to provide a patient interface device that overcomes the shortcomings of conventional interface devices. This object is achieved according to one embodiment of the present invention by providing a patient interface device where the material characteristics of headgear straps are used to implement auto-adjustment. This feature will allow a patient to place a patient interface device over the patient's head without having to disconnect or adjust headgear straps.

In addition, a patient interface device according to the invention will automatically adjust to and have enough pressure on a patient's face to seal the patient interface device to the patient's nose and/or mouth without having to manually adjust the headgear straps. This design feature will also prohibit a patient from over-tightening the headgear, which could cause red marks on the patient's scalp or face. In addition, the patient will have to make only minimal to no adjustments to the headgear for the patient interface device to work throughout the night.

In one embodiment of the invention, a patient interface device for delivering a flow of breathing gas to an airway of a patient is provided that includes headgear that optimizes the material properties of flexible polymeric materials to have enough pull force for a cushion on a mask to seal to the patient under a range of pressures. The thicknesses of the materials can be varied over the design profile to optimize the pull direction and pull force. Different materials of varying elasticity or durometer hardness will be used to also vary the stretch and optimize the design. The different materials will be joined by, for example, overmolding, suitable adhesives, or chemical, mechanical, or thermal bonding.

In another embodiment of the invention, an auto-adjusting strap/headgear system for use in a patient interface device comprises first and second lateral strap sections, each lateral strap section having a front portion and a rear portion, a rear strap section having distal ends connected to the rear portions of the lateral strap sections, and a top strap extending from the first lateral section to the second lateral section. The lateral strap sections each comprise material having a durometer hardness of from about 30 to about 85 Shore A, and the rear strap section comprises material having a durometer hardness of from about 3 Shore 000 to about 40 Shore A. The lateral strap sections, the top strap, and the rear strap section are configured so that the strap/headgear system can auto-adjust to fit a patient's head.

In another embodiment of the invention, a patient interface system for delivering a flow of breathing gas to an airway of a patient comprises a mask component and an auto-adjusting strap/headgear system which comprises first and second lateral strap sections, a rear strap section, and a top strap extending from the first lateral strap section to the second lateral strap section. Each lateral strap section has a front portion and a rear portion, and the rear strap section has distal ends connected to the rear portions of the lateral strap sections. The lateral strap sections each comprise material having a durometer hardness of from about 30 to about 85 Shore A, and the rear strap section comprises material having a durometer hardness of from about 3 Shore 000 to about 40 Shore A. The lateral strap sections, the top strap, and the rear strap section are configured so that the strap/headgear system can auto-adjust to fit a patient's head.

The present invention also contemplated providing a headgear in which a portion of the top strap, the rear strap, or both includes a high friction material disposed so as to contact a portion of the user responsive to the headgear being donned by a user. This feature of the present invention avoids or reduces slipping of the headgear on the user.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic lateral elevational view, and

FIG. 2 is a schematic rear elevational view, of a headgear system for a patient interface device adapted to provide a regimen of respiratory therapy to a patient according to one exemplary embodiment of the present invention;

FIG. 3 is a schematic lateral elevational view, and

FIG. 4 is a schematic rear elevational view, of a headgear system for a patient interface device adapted to provide a regimen of respiratory therapy to a patient according to another exemplary embodiment of the present invention;

FIGS. 5 and 6 are each a schematic rear elevational view of a headgear system for a patient interface device adapted to provide a regimen of respiratory therapy to a patient according to a further exemplary embodiment of the present invention;

FIG. 7 is a side view of a portion of further embodiment of a headgear system according to the principles of the present invention; and

FIG. 8 is a rear view of the portion of the headgear system shown in FIG. 7.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality). As employed herein, durometer hardness shall refer to Shore hardness as measured by a durometer.

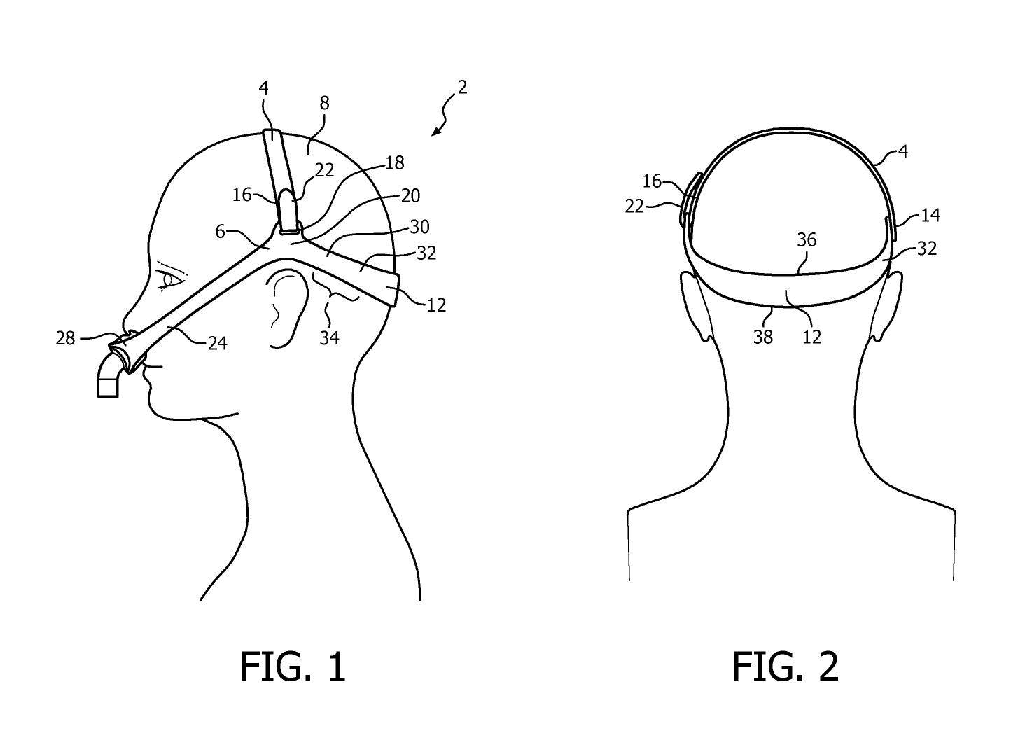

FIG. 1 is a schematic lateral elevational view, and FIG. 2 is a schematic rear elevational view, of an exemplary embodiment of the invention with an auto-adjusting strap/headgear system comprising lateral straps, a rear strap, and a fabric top strap. A strap system 2 comprises a top strap 4, a lateral strap section 6 on each side of a patient's head 8, and a rear strap section 12. Top strap 4 has distal ends 14, 16 that engage each lateral strap section 6 at an opening 18 in an attachment point or projection 20. On one side of wearer's head 8 distal end 14 is fixedly attached or engaged to an opening 18, and the other side distal end 16 comprises a releasable hook and loop system 22, such as a VELCRO® hook and loop system, that loops through an opening 18 and removably fixedly attaches to a lateral strap section 6. Optionally, distal end 14 also comprises a releasable hook and loop system 22, such as a VELCRO® hook and loop system, that loops through an opening 18 and removably fixedly attaches to a lateral strap section 6. A front section 24 of each lateral strap section 6 is removably or permanently attached to a mask component 28. Thus, top strap 4 is adjustable with regard to one or both lateral strap sections 6.

The rear portion 30 of each lateral strap 6 engages a distal portion 32 of rear strap section 12 at section 34. Section 34 may comprise a section where rear portion 30 is molded over distal portion 32, or vice versa, or rear portion 30 and distal portion 32 may be attached or bonded at section 34 by adhesives or in some other mechanical, thermal, or chemical manner.

Lateral strap sections 6 and rear strap section 12 will, in the exemplary embodiment, comprise material such as, without limitation, silicone. Lateral strap sections 6 in the exemplary embodiment comprise a high durometer material, that is, a less elastic material, for example, a material having a durometer hardness from about 30 to about 85 Shore A, or a 100% modulus of elasticity of from about 100 to about 1000 psi, to control stretch, whereas rear strap section 12 comprises low durometer, high elasticity material, for example, material having a durometer hardness of from about 3 Shore 000 to about 40 Shore A, or a 100% modulus of elasticity of from about 1 to about 200 psi.

The thickness of rear strap section 12 will vary to control stretch positions and properties. A thicker section will require more force to elongate whereas a thinner section will require less force to elongate the same distance. By determining the thickness and where the thick and thin sections are, elongation and direction can be controlled. For example, there may be a thin portion close to section 34 and thicker sections in the middle of rear strap section 12, giving the majority of the elongation at the side of the head. However, it may be necessary to have a thinner section in the middle of section 12 and thicker sections at section 34 to have greater elongation on or at the back of patient's head 8. The thickness of rear strap section 12 can also be controlled.

The top edge portion 36 of rear strap section 12 may be thinner to allow more elongation and adjustment over a patient's occipital bone (not shown), and the bottom edge portion 38 of rear strap section 12 will be thicker so that it will not elongate around patient's head 8. Alternatively, the middle of rear strap section 12 will be thicker than top edge portion 36 and bottom edge portion 38 to control the structure but allow top edge portion 36 and bottom edge portion 38 to custom fit around patient's head 8. One skilled in the art would appreciate that other variations in thickness and length will permit optimization. Top strap 4, in the exemplary embodiment, comprises a fabric such as standard 3-layer laminate (for example, LYCRA® foam, UBL).

The widths and thicknesses of strap sections 4, 6, and 12 can vary, according to desired characteristics and design features. For example, the thicknesses can be from about 0.5 mm to about 7 mm, and the widths can be from about 7 mm to about 20 mm.

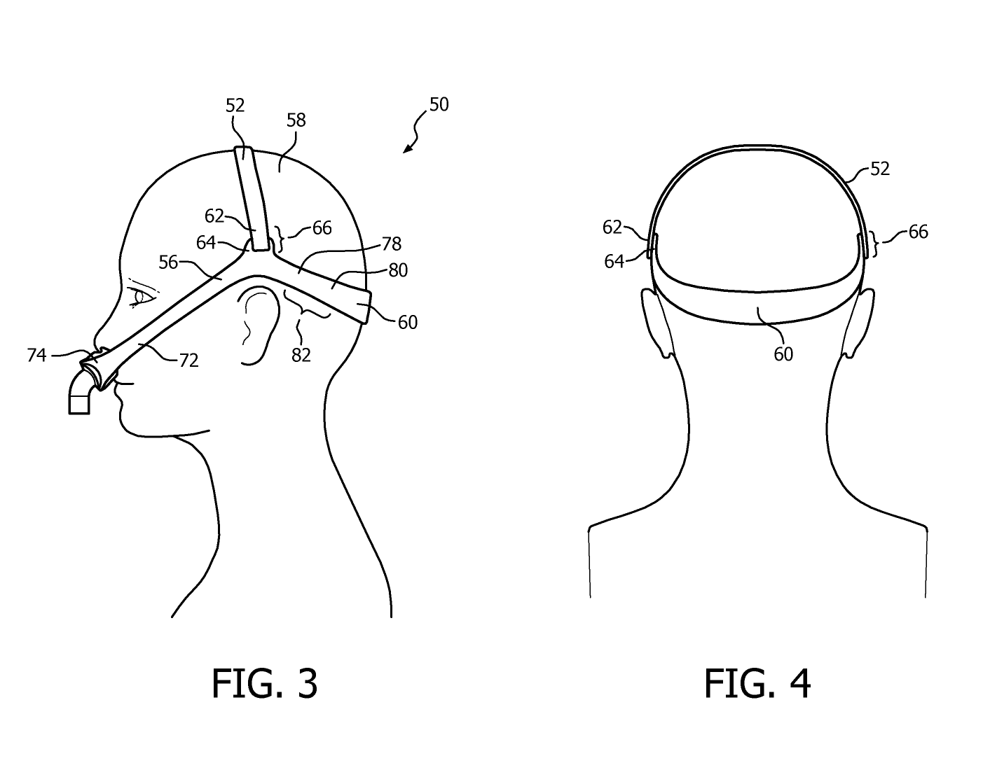

FIG. 3 is a schematic lateral elevational view, and FIG. 4 is a schematic rear elevational view, of an alternative exemplary embodiment of the invention with an auto-adjusting strap/headgear system comprising lateral straps, a rear strap and a top strap. A strap system 50 comprises a top strap 52, a lateral strap section 56 on each side of a patient's head 58, and a rear strap section 60. Top strap 52 has distal ends 62 that respectively are fixedly attached to or engage each lateral strap section 56 at an attachment member or projection 64 at a section 66. Section 66 may comprise a section where each distal end 62 is molded over projection 64, or vice versa, or each distal end 62 and projection 64 may be attached or bonded at section 66 by adhesives or in some other mechanical, thermal, or chemical manner. A front section 72 of each lateral strap section 56 is removably or permanently attached to a mask component 74.

The rear portion 78 of each lateral strap section 56 engages a distal portion 80 of rear strap section 56 at an overmold section 82. Overmold section 82 may comprise a section where rear portion 78 is molded over distal portion 80, or vice versa, or rear portion 78 and distal portion 80 are attached or bonded at section 82 by adhesives or in some other mechanical, thermal, or chemical manner.

Rear strap section 60 in this exemplary embodiment will comprise material such as, without limitation, silicone, with high elasticity/elongation characteristics having a durometer hardness of, for example, from about 3 Shore 000 to about 40 Shore A, or a 100% modulus of elasticity of from about 1 to about 200 psi, that is, it will be “stretchy”. Lateral strap sections 56 comprise less elastic material such as, without limitation, silicone, having, for example, a durometer hardness of from about 30 to about 85 Shore A, or a 100% modulus of elasticity of from about 100 to about 1000 psi, to control stretch.

Top strap 52 may comprise a combination of low elongation/low elasticity silicone of durometer hardness of, for example, from about 30 to about 85 Shore A, or a 100% modulus of elasticity of from about 100 to about 1000 psi, at ends 62 and high elongation/high elasticity silicone of durometer hardness of, for example, from about 3 Shore 000 to about 40 Shore A, or a 100% modulus of elasticity of from about 1 to about 200 psi, in the middle. The thicknesses will vary to control stretch positions and properties. A thicker section will require more force to elongate whereas a thinner section will require less force to elongate the same distance. By determining the thickness and where the thick and thin sections are, elongation and direction can be controlled.

The widths and thicknesses of strap sections 52, 56, and 60 can vary, according to desired characteristics and design features. For example, the thicknesses can be from about 0.5 mm to about 7 mm, and the widths can be from about 7 mm to about 20 mm.

Mask components 28 and 74 can be any mask device for providing respiratory therapy. Such devices include, without limitation, a nasal mask that covers a patient's nose, a nasal cushion having nasal prongs that are received within a patient's nares, a nasal/oral mask that covers a patient's nose and mouth, and a full face mask that covers a patient's face.

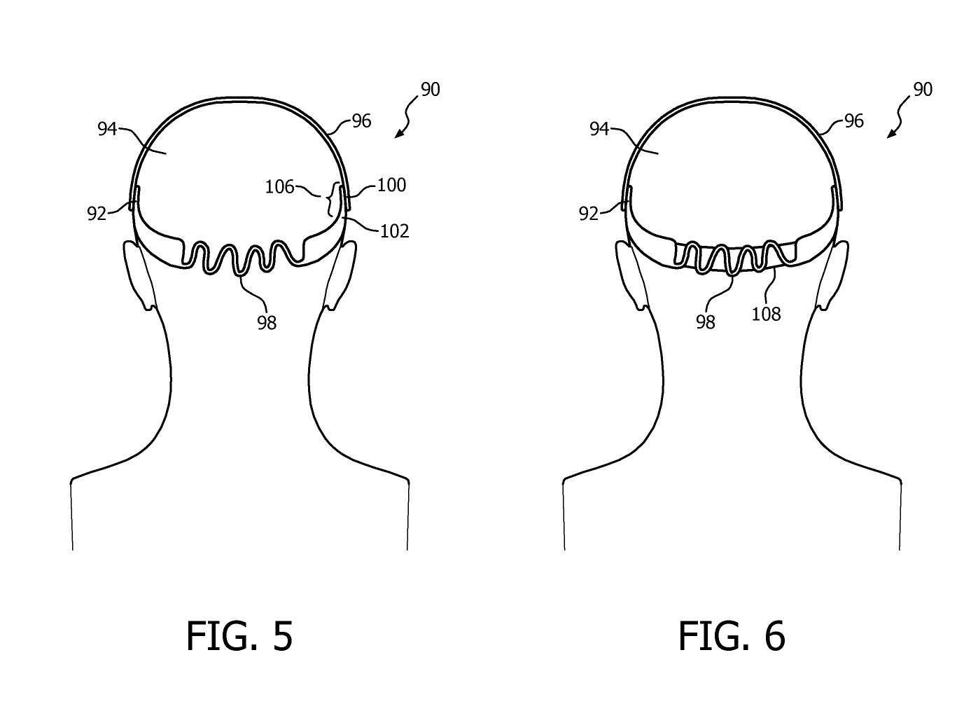

FIGS. 5 and 6 are each a schematic rear elevational view of an embodiment of the invention with an auto-adjusting strap/headgear system comprising lateral straps, a top strap, and a Z-design back strap. A strap system 90 comprises a lateral strap section 92 on each side of a patient's head 94, a top strap 96, and a rear strap section 98. The rear portion 100 of each lateral strap section 92 is attached to a distal portion 102 of rear strap section 98 at an overmold section 106 through overmolding, adhesives, or mechanical, thermal, or chemical attachment, as described above. Lateral strap section 92 will be made from a silicone. Top strap 96 can be either a fabric strap such as described above for top strap 4 or a variably elastic strap such as described for top strap 52. Similarly, top strap 96 can engage lateral strap sections 92 in the same fashion as described for the embodiments set forth above in FIGS. 1 and 2 and FIGS. 3 and 4, that is, fixedly or with a hook and loop arrangement.

Rear strap section 98 is a Z-shaped silicone or thermoplastic member comparable to a well-known Goody's hair band, that would allow lateral strap sections 92 to expand and contract to fit patient's head 94 comfortably. The Z-shaped design creates a spring force that will pull the headgear tightly to patient's head 94. As shown in FIG. 6, there can optionally be a silicone webbing 108 attached to Z-shaped section 98 to prevent hair from becoming entangled in strap section 98. Silicone webbing 108 will be thin as compared to the thickness of Z-shaped section 98.

Lateral strap sections 92 in this exemplary embodiment comprise lower elongation/elasticity material such as, without limitation, silicone, with a durometer hardness of, for example, from about 30 to about 85 Shore A, or a 100% modulus of elasticity of from about 100 to about 1000 psi, to control stretch. Rear strap section 98 comprises higher elongation/elasticity material such as, without limitation, silicone, having a durometer hardness of from about 3 Shore 000 to about 40 Shore A, or a 100% modulus of elasticity of from about 1 to about 200 psi. The thicknesses will vary to control stretch positions and properties, as described above. Preferably silicone webbing 108 will have the same elasticity/elongation characteristics as rear strap section 98.

The widths and thicknesses of strap sections 92, 96, and 98 can vary, according to desired characteristics and design features. For example, the thicknesses can be from about 0.5 mm to about 7 mm, and the widths can be from about 7 mm to about 20 mm.

The preferred polymeric materials useful herein are thermoplastic elastomers, such as polyurethanes, or silicones, that are readily commercially available. Examples of such silicones include Wacker 3003/3009 family of silicones, available from Wacker Chemie AG, Munich, Germany, Bluestar 4310 silicone, available from Bluestar Silicones USA Corp., East Brunswick, N.J., and Shin Etsu 2090 family of silicones, available from Shin Etsu Chemical Co., Ltd., Tokyo.

In the description above it is indicated that certain sections can be overmolded to join such sections together. This process usually includes one material (material X) being molded first into the desired form, and then, once material X has begun to cure from liquid to solid, the next material (material Y) can be molded on top of material X at certain areas, creating cross-linked material and/or chemically bonded materials. As one skilled in the art would appreciate, other methods and techniques for bonding polymeric sections together can be used, including, but not limited to, bonding by chemical, mechanical, or thermal means. Mechanical interlocks can be designed into material X structure (i.e., holes), and when material Y is molded, the uncured material will flow in and around material X structure to create a mechanical interlock. This process is seen on current Philips Respironics mask such as Comfort Gel Full Silicone Flap overmolded to a thermoplastic retaining ring. In instances where materials are chemically bonded, adhesives such as, without limitation, LSR, UV cure adhesive, instant adhesive, RTV, RTV2, can be used to bond material X to material Y.

In one embodiment of the invention, a patient interface device for delivering a flow of breathing gas to an airway of a patient is provided that includes headgear that optimizes the material properties of flexible polymeric material to have enough pull force for a cushion on a mask to seal to the patient under a range of pressures. The thicknesses of the materials can be varied over the design profile to optimize the pull direction and pull force. Different materials of varying elasticity will be used to also vary the stretch and optimize the design. The different materials will be joined by, for example, overmolding or suitable adhesives or bonding, as described herein.

In another embodiment of the invention, controlling the texture of the headgear straps by applying coatings to the straps allows optimization of headgear design. For example, a parylene coating can be vapor deposited on one or more straps. Such a coating will provide a silky texture due to its low coefficient of friction. Other examples include topcoat materials available from Momentive Performance Products, of Albany, N.Y., or NuSil Silicones, of Carpinteria, Calif., that can be sprayed on.

Uncoated silicon material will tend to be sticky, and coating such areas with a coating such as parylene will result in respective areas of stickiness and smoothness. Causing certain areas to be sticky and other areas to be smooth enhances design by increasing stability of the mask and preventing the headgear from slipping on a patient's hair.

Another manner of controlling the texture of different surfaces of material, that is, whether sticky or smooth, is to apply different surface textures to a mold that may enhance the “stickiness” of the material or the smoothness. For example, laser etching, VDI (EDM), chemical etching, sandblasting, and the like, may have this effect. Lastly, another method of controlling surfaces finish of material, that is, whether sticky or smooth, is to use a blooming agent within the material. Blooming agents are additives such as a colorant that causes a silicone member to feel “silky” or smooth to the touch. The stickiness and smoothness can be controlled by covering areas that are desired to be sticky and not allowing the blooming agent to cure. If a blooming agent is not used, a coating can be applied to the materials to control the stickiness.

In an exemplary embodiment of the present invention at least a portion of the headgear includes a material with a high coefficient of friction to help anchor that portion of the headgear on the head. For example, the present invention contemplates providing the headgear back strap or a portion thereof with a high friction material to anchor the back strap around the back of the head. This feature of the present invention (i.e., providing a high friction surface on a strap or a portion of a strap) can be used alone or in combination with the auto-adjusting feature discussed above.

In one embodiment, a high friction material is applied to the inside surface of the back strap that contacts the patient. The application of high friction material, such as a foam, on the surface of the back strap enhances friction between the back strap and the patient. Increased coefficient of friction improves grip of back strap on back of head. This aids back strap in maintaining position and improves stability of headgear. This also reduces sensation of movement and need to adjust the mask during use, which disturbs the patient's sleep.

It can be appreciated that providing a high friction material to the inside surface of the back strap or a portion thereof reduces movement of the back strap, which contributes to instability of the mask possibly contributing to leak. Movement of the back strap component typically yields movement of the mask. This can require readjustment of the mask during use, which is disruptive the patient sleep. The back strap can also slide up and off the head. Movement of the back strap may also result in a sensation of movement that annoys or disturbs the patient. The cases may not result in leak. However, these results can disrupt the patient's sleep.

Often, rigid elements are incorporated into designs utilizing hard materials to provide structure in an attempt to provide a more stable platform. Others may simply utilize elastic, which can aid fit and grip. However, these are not very effective in reducing slip of the back strap. The present invention addresses provides the following benefits:
Soft and Compliant—the back strap will be soft to the touch and compliant, allowing it to conform to the patient's head.
Reduce Slipping—the back strap will reduce slipping, maintaining its position better than current design that utilize woven or knit fibers contacting the patient.
Stability—Reducing movement of the back strap can improve mask stability
Reduce Leak—Improving stability will help reduce leaks.
Less Disruption—Reducing movement of the back strap will help reduce disruption of the patient's sleep. This is accomplished by mitigating the need to adjust the mask during use and mitigating the sensation of movement, which can disturb the patient.

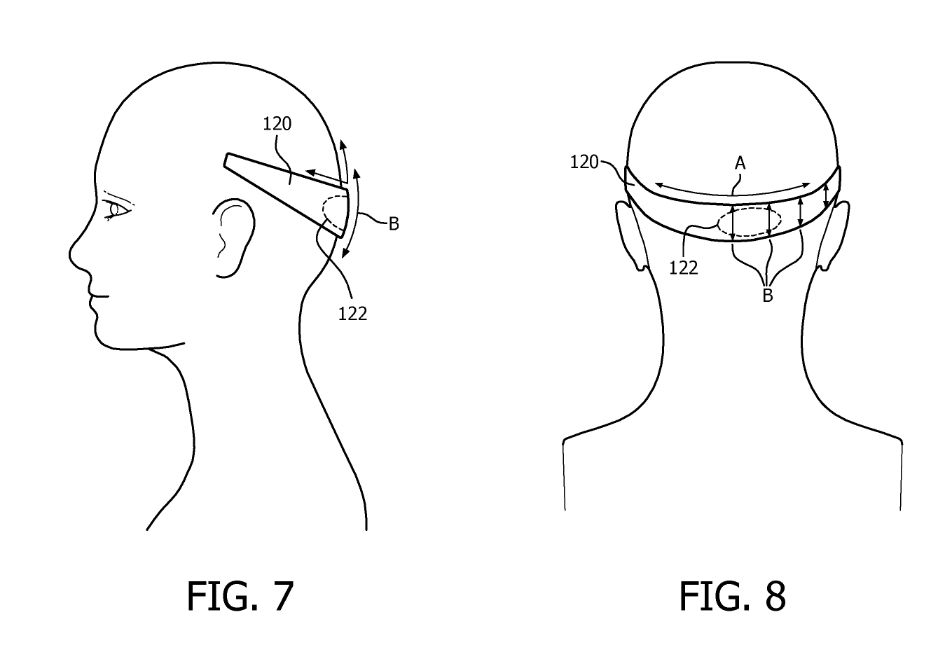

FIGS. 7 and 8 illustrate a portion of a proposed headgear back strap 120 that is soft and flexible, but also provides the proper stability to maintain seal. In an exemplary embodiment, this is achieved by a combination of elements forming the headgear. The back strap of the headgear has tensional forces, as indicated by arrow A, acting upon it that can result in movement and possibly pulling it upward out of position. These forces are primarily generated by the strapping force of the mask. However, the back strap may also have forces pulling on it, as indicated by arrows B, due, for example, to contact with the patient's bed or bed pillow. A high friction material 122 is located on the back strap so as to contact the back of the patient's head in the area of the occipital bone or slightly above that location. Of course, the present invention contemplates that the back strap can be formed such that the entire patient-contacting portion include the high-coefficient material. In this embodiment, material 122 has a higher coefficient of friction than an adjacent portion of the headgear.

Materials having a coefficient of friction that is higher than typical fabrics will aid the back strap component in gripping the patient's head and mitigate movement. Applying materials, such as Thermoplastic Elastomers (TPE), such as PE, PP, or silicones, to the headgear or portion thereof provides resistance to movement. Such materials can be applied in a form of foam. This foam can be applied to the material in multiple ways, including but not limited to lamination, RF Welding, thermoforming, and coatings. The structure under this particular design can include a lamination of woven or knitted filaments (fibers) to provide support.

The present invention further contemplates that the back strap or other portions of the headgear will also conform to the patient's head, as shown in FIGS. 7 and 8 and is flexible. This aids in comfort and ability to grip, securing the location. In an exemplary embodiment, the headgear will utilize a foam material as the high friction material. This material provides a soft texture, compressible and flexible layer with a high coefficient of friction.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An auto-adjusting strap/headgear system for use in a patient interface device, which strap/headgear system comprises:
first and second lateral strap sections, each lateral strap section having a front portion and a rear portion, wherein the lateral strap sections each comprise a first durometer material having a first durometer hardness of from about 30 to about 85 Shore A and a first 100% modulus of elasticity of from about 100 to about 1000 psi;
a rear strap section having two distal ends connected to the rear portions of the lateral strap sections, wherein the rear strap section comprises a second durometer material having a second durometer hardness different than the first durometer hardness and being of from about 3 Shore 000 to about 40 Shore A and a second 100% modulus of elasticity being different than the first 100% modulus of elasticity and being of from about 1 to about 200 psi; and
a top strap having distal ends that engage the first and second lateral strap sections, wherein the lateral strap sections, the top strap, and the rear strap section are configured so that the strap/headgear system can auto-adjust to fit a patient's head.

2. The auto-adjusting strap/headgear system according to claim 1, wherein each rear portion of a lateral strap section is molded over a distal end of the rear strap section.

3. The auto-adjusting strap/headgear system according to claim 1, wherein each distal end of the rear strap section is molded over a rear portion of a lateral strap section.

4. The auto-adjusting strap/headgear system according to claim 1, wherein each rear portion of a lateral strap section is bonded to a distal end of the rear strap section by adhesives or other chemical, mechanical, or thermal means.

5. The auto-adjusting strap/headgear system according to claim 1, wherein the top strap comprises fabric and wherein the distal ends of the top strap attach to the lateral strap sections.

6. (canceled)

7. The auto-adjusting strap/headgear system according to claim 1, wherein each lateral strap section has an attachment point with an opening to which a distal end of the top strap attaches.

8. (canceled)

9. The auto-adjusting strap/headgear system according to claim 1, wherein the top strap comprises low elongation/low elasticity silicone of durometer hardness of from about 30 to about 85 Shore A at its distal ends and high elongation/high elasticity silicone of durometer hardness of from about 3 Shore 000 to about 40 Shore A between the distal ends.

10. The auto-adjusting strap/headgear system according to claim 9, wherein the each distal end of the top strap attaches to a projection member on a lateral strap member.

11. The auto-adjusting strap/headgear system according to claim 10, wherein each distal end of a top strap is molded over a projection member of a lateral strap section.

12. The auto-adjusting strap/headgear system according to claim 10, wherein each projection member of a lateral strap section is molded over a distal end of a top strap.

13. (canceled)

14. The auto-adjusting strap/headgear system according to claim 1, wherein the rear strap section has a Z-design.

15. The auto-adjusting strap/headgear system according to claim 14, wherein the rear strap section has a web underneath the rear strap section.

16. The auto-adjusting strap/headgear system according to claim 1, wherein at least a portion of the top strap, the rear strap, or both includes a high friction material disposed so as to contact a portion of the user responsive to the headgear being donned by a user.

17. An patient interface system for delivering a flow of breathing gas to an airway of a patient, which comprises:
(a) a mask component; and
(b) an auto-adjusting strap/headgear system according to claim 1.

18. (canceled)

19. The auto-adjusting strap/headgear system according to claim 1, the rear strap section has a first portion having a first coefficient of friction and a second portion having a second coefficient of friction greater than the first coefficient of friction.
